(19) United States
(12) Patent Application Publication
(10) Pub. No.: US 2009/0036831 A1
(43) Pub. Date: Feb. 5, 2009

(54) BALLOON CATHETER WITH DELIVERY PORTS

(75) Inventor: William L. Howat, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/832,277

(22) Filed: Aug. 1, 2007

(51) Int. Cl. A61M 25/10

(52) U.S. Cl. 604/102.01

(57) ABSTRACT

The present invention provides apparatus and methods for treating a vascular condition by providing a catheter having proximal and distal regions, a balloon disposed on the distal region, and a plurality of delivery ports disposed in lateral surfaces of the catheter at locations proximal to the balloon. The balloon is adapted to treat the vascular condition, for example, by performing balloon angioplasty. Subsequently, the balloon may be deflated, the catheter advanced distally, and the plurality of delivery ports may be aligned with the vascular condition, e.g., to deliver a therapeutic agent such as an anti-restenosis drug. During the delivery of the therapeutic agent, the balloon may be partially or fully re-inflated to enhance localized delivery of the therapeutic agent.

### BACKGROUND

The present invention relates generally to apparatus and methods for treating vascular conditions, and more specifically, to a catheter configured for balloon expansion of the vascular condition.

Atherosclerosis and other occlusive diseases are prevalent among a significant portion of the population. In such diseases, atherosclerotic plaque forms within the walls of the vessel and blocks or restricts blood flow through the vessel. Atherosclerosis commonly affects the coronary arteries, the aorta, the iliofemoral arteries and the carotid arteries. Several serious conditions may result from the restricted blood flow, such as ischemic events.

Various procedures are known for treating stenoses in the arterial vasculature, such as balloon angioplasty. During a balloon angioplasty procedure, a catheter having a deflated balloon attached thereto is inserted into a patient's vessel. Once positioned across a constricting lesion, the balloon is then inflated to widen the lumen to partially or fully restore patency to the vessel. After satisfactory widening of the stenosis has been achieved, the balloon is deflated. The catheter is then retracted and removed from the patient's vessel with the balloon in the deflated state.

One problem that exists with conventional balloon angioplasty techniques is that after treatment is applied and patency is temporarily restored, a subsequent narrowing of the vessel, or “restenosis,” may occur. While the exact rates are not known, the instances of restenosis after balloon angioplasty may be as high as about 35%.

Various techniques have been used to reduce the likelihood of restenosis after a balloon angioplasty procedure. For example, stenting is one exemplary anti-restenosis technique that involves the insertion of a usually tubular member into a vessel to help maintain patency. Further, various stents have been coated using therapeutic agents, such as drugs or bioactive materials, to achieve a biological effect in addition to applying a radially outward force. Such drug coated stents may deliver the agents in close proximity to a stenotic lesion to reduce the likelihood of restenosis.

Still other methods and apparatus have been developed in an attempt to reduce restenosis rates, including multiple inflations of the balloon, performing atherectomy procedures, using lasers to treat the condition, or infusing a fluid or agent through one or more delivery ports in the vicinity of the vascular condition.

In view of the above, it would be desirable to provide an apparatus and method to treat a vascular condition, such as a stenotic lesion, that employs multiple therapeutic approaches in one easy-to-use device in order to reduce the likelihood of restenosis.

### SUMMARY

The present invention provides apparatus and methods for treating a vascular condition by providing a catheter having a balloon and a plurality of delivery ports disposed in lateral surfaces of the catheter at locations proximal to the balloon. The balloon is adapted to treat the vascular condition, for example, by performing balloon angioplasty, and the plurality of delivery ports may be used to subsequently deliver a therapeutic agent, such as an anti-restenosis drug, to the site of the vascular condition.

In a first embodiment, the balloon is disposed on a distal region of the catheter. After the balloon treats the vascular condition, the balloon may be deflated, the catheter may be advanced in a distal direction, and the plurality of delivery ports may be substantially aligned with the vascular condition. At this time, the balloon may be partially or fully re-inflated at a location distal to the vascular condition. A therapeutic agent may then be delivered to the vascular condition via the plurality of delivery ports. Since the balloon is partially or fully inflated distal to the vascular condition, enhanced localized delivery of the therapeutic agent to the vascular condition may be achieved.

In a preferred embodiment, a wire guide lumen may extend between proximal and distal regions of the catheter, and the plurality of delivery ports may be placed in fluid communication with the wire guide lumen. In use, the wire guide lumen receives a wire guide therein, and permits the injection of the therapeutic agent in an annular space formed between the wire guide and the catheter. This allows the injection of the therapeutic agent through the wire guide lumen and the plurality of delivery ports.

Therefore, the apparatus and methods allow a two-pronged approach to treating a vascular condition, i.e., by performing balloon dilation of the vascular condition, followed by subsequent localized injection of therapeutic agents to the target site. Preferably, a longitudinal length spanned by the plurality of delivery ports along the catheter is substantially identical to a longitudinal length spanned by a treatment section of the balloon. The substantially identical longitudinal lengths may correspond generally to the length of the vascular condition to facilitate treatment.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a side view of a catheter that may be used to treat a vascular condition.

FIG. 2 is a cross-sectional view taken along line A-A of FIG. 1.

FIG. 3 is an enlarged view of a distal portion of the apparatus of FIG. 1.

FIGS. 4A-4D illustrate method steps that may be used to treat a vascular condition, and depict side-sectional views of a vessel and side views of apparatus disposed therein.

### DETAILED DESCRIPTION OF THE PREFERRED EMBODIMENTS

In the present application, the term “proximal” refers to a direction that is generally toward a physician during a medical procedure, while the term “distal” refers to a direction that is generally toward a target site within a patient's anatomy during a medical procedure.

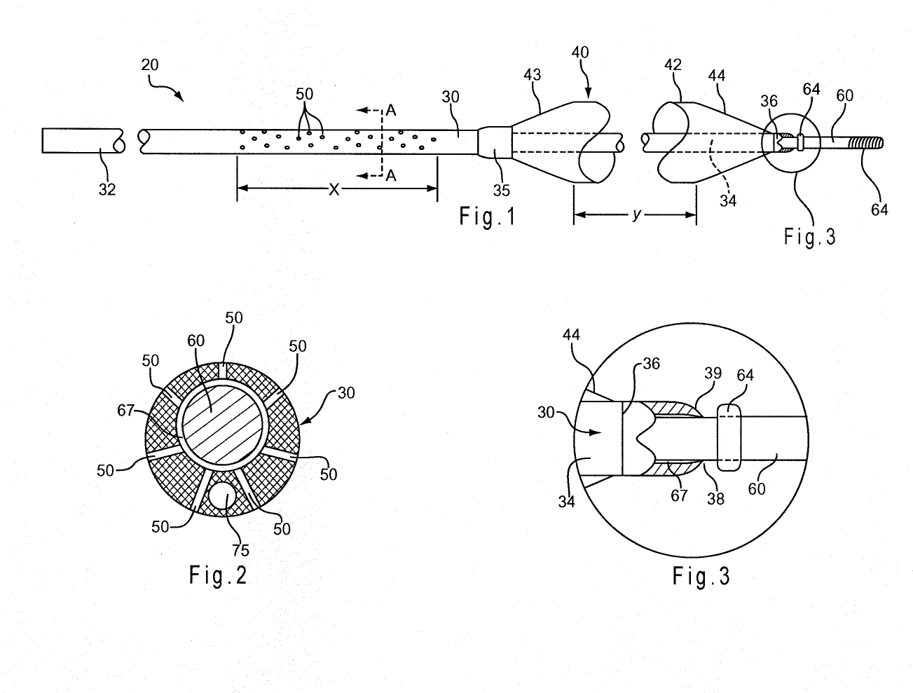

Referring now to FIGS. 1-3, an apparatus suitable for treating a vascular condition, such as a stenosis within a vessel, is described. Apparatus 20 comprises catheter 30, balloon 40 and a plurality of delivery ports 50, as shown in FIG. 1. Catheter 30 has proximal and distal regions 32 and 34, and balloon 40 preferably is disposed on distal region 34, as shown in FIG. 1. As will be explained in further detail below, the plurality of delivery ports 50 may be disposed in lateral surfaces of catheter 30 at locations proximal to balloon 40, and may be used to deliver one or more therapeutic agents to the site of the vascular condition.

Catheter 30 may comprise a flexible, tubular member that may be formed from one or more semi-rigid polymers. For example, the catheter may be manufactured from polyurethane, polyethylene, tetrafluoroethylene, polytetrafluoroethylene, fluorinated ethylene propylene, nylon, PEBAX or the like.

As shown in FIG. 1, balloon 40 may comprise proximal taper 43, distal taper 44, and treatment section 42 disposed therebetween. Proximal and distal tapers 43 and 44 may be attached to catheter 30 at proximal and distal attachment regions 35 and 36, respectively. Balloon 40 may be attached at these locations using any suitable adhesive, such as a biocompatible glue, or alternatively, using heat-shrink tubing, heat bonding, laser bonding, welding, solvent bonding, one or more tie-down bands, or the like. Balloon 40 may be manufactured from a balloon material, e.g., PEBAX, nylon, Hytrel, Arnitel or other polymers, which may be suitable for use during an interventional procedure.

Catheter 30 preferably comprises wire guide lumen 67, which is configured to receive wire guide 60. As shown in FIG. 2, an outer diameter of wire guide 60 may be smaller than a diameter of wire guide lumen 67, thereby forming an annular space between wire guide 60 and catheter 30. The plurality of delivery ports 50 may be disposed in multiple lateral surfaces of catheter 30, e.g., around the perimeter of catheter 30, and may be placed in fluid communication with wire guide lumen 67, as shown in FIG. 2. As will be set forth in further detail below, when one or more therapeutic agents are delivered through the annular space between wire guide 60 and catheter 30, the therapeutic agents may be delivered to the site of the vascular condition via the plurality of delivery ports 50.

As shown in FIG. 2, catheter 30 may comprise inflation lumen 75, which may span from proximal region 32 to distal region 34 of catheter 30. At proximal region 32, inflation lumen 75 may be coupled to an inflation source. At distal region 34, at least one side port may be disposed in a lateral surface of catheter 30 to provide fluid communication between inflation lumen 75 and the inner confines of balloon 40, to thereby allow for inflation and deflation of balloon 40.

Inflation lumen 75 may be integrally formed with catheter 30, e.g., by extrusion. Alternatively, a separate inflation lumen may be provided, for example, using an additional piece of tubing disposed on an external surface of catheter 30, whereby the piece of tubing is placed in fluid communication with balloon 40.

In the embodiment of FIGS. 1-3, inflation lumen 75 may be disposed between two delivery ports 50, as shown in FIG. 2, but is not placed in fluid communication with wire guide lumen 67. Therefore, therapeutic agents that are delivered through delivery ports 50 may be separated from fluid flowing in inflation lumen 75.

Catheter 30 and/or wire guide 60 may comprise features that prevent the flow of therapeutic agents delivered via wire guide lumen 67 from exiting through distal tip 38 of catheter 30, thereby ensuring that substantially the entire amount of therapeutic agent that is delivered exits through delivery ports 50. For example, as shown in FIG. 3, distal tip 38 of catheter 30 may comprise inward taper 39, which may cause distal tip 38 to fit snugly around an exterior surface of wire guide 60. Inward taper 39 may be configured to permit distal advancement of catheter 30 over wire guide 60, while substantially inhibiting flow of fluid through distal tip 38 of catheter 30. Optionally, wire guide 60 may be coated with a substance, such as polytetrafluoroethylene, which may enhance the sealing ability between distal tip 38 of catheter 30 and wire guide 60.

As a further option, wire guide 60 may comprise bead element 64, which may be disposed on the distal region of wire guide 60 and comprise a slightly larger outer diameter relative to proximal segments of wire guide 60, as shown in FIG. 3. In use, when catheter 30 is advanced distally over wire guide 60 and abuts bead member 64, a substantially fluid tight seal may be formed to prohibit the flow of the therapeutic agent through distal tip 38 of catheter 30.

In a further alternative embodiment, a circumferential sealing gasket may be affixed within the confines of wire guide lumen 67 at a location distal to delivery ports 50, e.g., in the vicinity of proximal attachment region 35. The sealing gasket may fill the annular space between wire guide 60 and catheter 30, such that wire guide 60 may be advanced through the sealing gasket, but fluid will not be allowed to flow distal to the gasket.

Referring back to FIG. 1, the plurality of delivery ports 50 may be disposed circumferentially and axially along a portion of catheter 30. The axial distance spanned by the collective plurality of delivery ports 50 has a longitudinal length “x.” Further, as shown in FIG. 1, treatment section 42 of balloon 40 spans a longitudinal length “y.” In a preferred embodiment, longitudinal length x of the delivery port region is substantially identical to longitudinal length y of balloon treatment section 42, for purposes that will be explained in greater detail below.

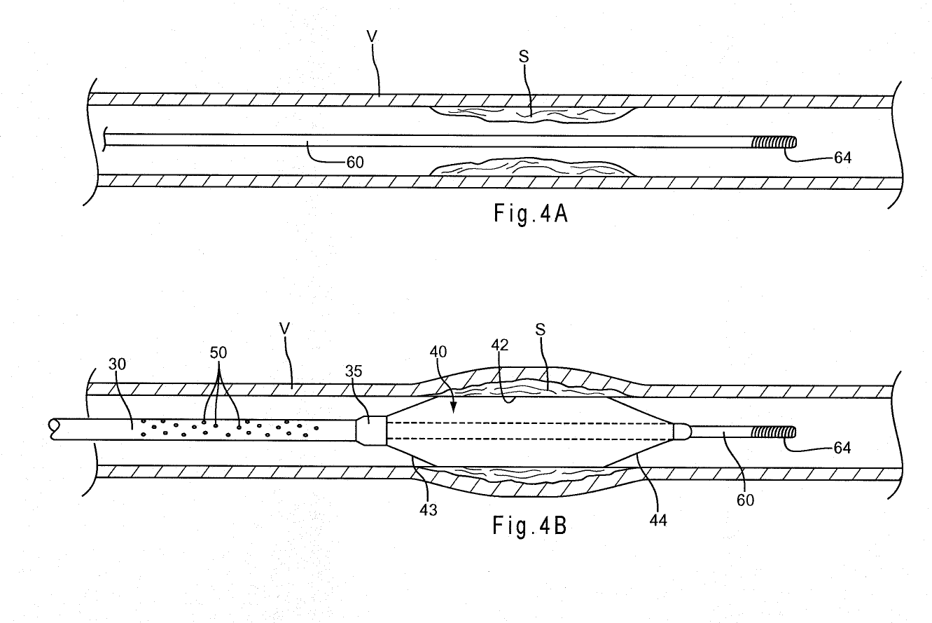

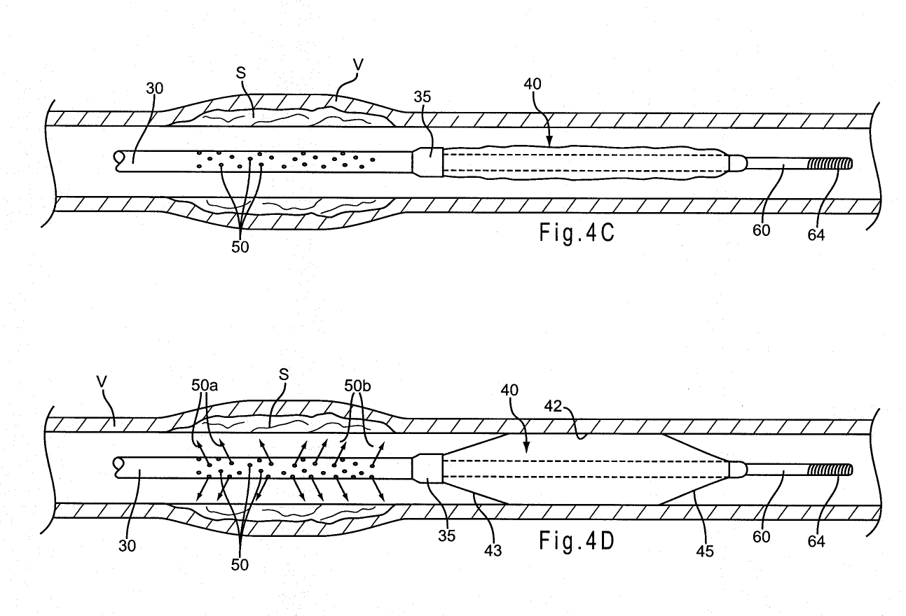

Referring now to FIGS. 4A-4D, a method for using apparatus 20 to treat a vascular condition, such as stenosis S in vessel V, is described. In a first step, wire guide 60 is advanced distally through a patient's vasculature. A coiled member 64 may be disposed at or near the distal end of wire guide 60 to provide an atraumatic tip and facilitate navigation through the vasculature. As shown in FIG. 4A, the distal end of wire guide 60 traverses stenosis S, preferably using fluoroscopic guidance, and is disposed distal to the stenosis.

In a next step, catheter 30 is advanced distally over wire guide 60 with balloon 40 in an uninflated state. Balloon 40 is aligned with stenosis S in the uninflated state. One or more radiopaque markers (not shown) may be disposed on catheter 30, preferably in a region underlying balloon 40, to facilitate positioning of balloon 40 with respect to stenosis S. When properly aligned, an inflation fluid, such as saline, may be injected through inflation lumen 75, through aperture 76, and into the inner confines of balloon 40 to inflate the balloon. As shown in FIG. 4B, treatment section 42 of balloon 40 performs balloon angioplasty on stenosis S, thereby dilating the stenosis and partially or fully restoring patency within a diseased section of vessel V.

Optionally, one or more small perforations (not shown) may be formed in the distal end of catheter 30 or balloon 40 to permit oxygenated fluid to flow upstream to arterial vasculature during treatment of stenosis S. The oxygenated fluid may also be the balloon dilation fluid.

Referring now to FIG. 4C, in a next step, balloon 40 is deflated. Subsequently, apparatus 20 is advanced distally so that delivery ports 50 are substantially aligned with stenosis S. Catheter 30 may comprise one or more radiopaque markers disposed along the longitudinal length x of the delivery port region, to facilitate alignment of the delivery ports 50 with stenosis S. At this time, i.e., prior to infusion of therapeutic agents, catheter 30 may abut optional bead member 64 of wire guide 60 (see FIG. 3) to provide a fluid tight seal between distal tip 38 of catheter 30 and wire guide 60. Alternatively, other means for providing a fluid tight seal between distal tip 3 8 of catheter 30 and wire guide 60 may be employed, as discussed above.

Referring now to FIG. 4D, with delivery ports 50 substantially aligned with stenosis S, balloon 40 may be partially or fully re-inflated at a location distal to stenosis S. At this time, a therapeutic agent may be delivered through wire guide lumen 67. The therapeutic agent may flow in the annular space between wire guide 60 and catheter 30, as shown in FIG. 2, and as generally described above. The injected therapeutic agent flows through delivery ports 50 and is injected into vessel V in the vicinity of stenosis S, as shown in FIG. 4. Since balloon 40 has been temporarily re-inflated at a location distal to the stenosis, enhanced localized delivery of the therapeutic agent may be achieved, i.e., the therapeutic agent may be temporarily held in the vicinity of stenosis S upon ejection from delivery ports 50.

It should be noted that balloon 40 need not be fully re-inflated to occlude vessel V during injection of the therapeutic agent. Rather, partial inflation of balloon 40 may be provided, i.e., whereby the treatment section 42 of balloon 40 may not contact the intima of vessel V, thereby providing some level of interference to enhance localized drug delivery, while still allowing some blood flow to perfuse vessel V distal to balloon 40.

If necessary, any of the steps described in FIGS. 4B-4D may be repeated, and the same or an additional therapeutic agent may be subsequently delivered. Once satisfactory treatment of stenosis S has been achieved, catheter 30 and wire guide 60 may be removed from the patient's vessel with balloon 40 in the deflated state.

Advantageously, the apparatus and methods described above allow a two-pronged approach to treating a vascular condition, such as stenosis S, by allowing dilation of the stenosis using balloon 40, followed by subsequent localized injection of therapeutic agents to the target site. Since balloon 40 is disposed distal to delivery ports 50, and is partially or fully re-inflated at a location distal to the stenosis after dilating the stenosis, enhanced localized drug delivery may be achieved. Moreover, prior to the procedure, an individualized catheter may be selected, whereby delivery port treatment length x and balloon treatment section y may be sized and selected to treat a particular stenosis S. For example, by providing a plurality of delivery ports and a balloon having treatment zones that are substantially identical to one another, and substantially identical to the length of the vascular condition within the vessel, enhanced treatment of the vascular condition may be achieved.

In one embodiment, the plurality of delivery ports 50 may be angled in different directions. For example, one or more proximal delivery ports may be angled to eject fluid in a proximal direction, as shown by stream 50a in FIG. 4D. Similarly, one or more distal delivery ports may be angled to eject fluid in a distal direction, as shown by stream 50b in FIG. 4D. Alternatively, some of the proximal delivery ports may eject fluid in a distal direction, and or some of the distal delivery ports may eject fluid in a proximal direction. Any number of combinations is possible. The delivery ports may be curved or otherwise angled with respect to a longitudinal axis of catheter 30 to enable such directional delivery.

Further, it should be noted that, after performing the step described in FIG. 4D, a stenting procedure may be performed to further reduce the risk of restenosis. The stent may be delivered using a separate delivery apparatus after catheter 30 has been completely removed from vessel V. Alternatively, a stent introducer may be advanced co-axially over catheter 30 while apparatus 20 remains in place within vessel V.

As a further alternative approach, a stent may be coupled to balloon 40, e.g., by crimping the stent onto the balloon, and inserted along with balloon 40. The stent then is deployed when balloon 40 is first inflated, as shown in FIG. 4B. In this manner, the therapeutic agent may be delivered after the stent has been deployed to treat stenosis S.

It will be apparent that while the invention has been described primarily with respect to treatment of a stenosis within a vessel, the present invention may be used in other applications. Further, catheter 30 and wire guide 60 may employ an over-the-wire arrangement, as generally shown in FIG. 1, or alternatively, catheter 30 may comprise a rapid exchange port disposed in lateral surface of catheter 30 at a location proximal to delivery ports 50.

Moreover, the therapeutic agents used in conjunction with apparatus 20 may be chosen to perform a desired function upon ejection from delivery ports 50, and may be tailored for use based on the particular medical application. For example, the therapeutic agent can be selected to treat indications such as coronary artery angioplasty, renal artery angioplasty, carotid artery surgery, renal dialysis fistulae stenosis, or vascular graft stenosis. The therapeutic agent may be delivered in any suitable manner and in any suitable medium. The therapeutic agent may be selected to perform one or more desired biological functions, for example, promoting the ingrowth of tissue from the interior wall of a body vessel, or alternatively, to mitigate or prevent undesired conditions in the vessel wall, such as restenosis. Many other types of therapeutic agents may be used in conjunction with apparatus 20.

The therapeutic agent employed also may comprise an antithrombogenic bioactive agent, e.g., any bioactive agent that inhibits or prevents thrombus formation within a body vessel. Types of antithrombotic bioactive agents include anticoagulants, antiplatelets, and fibrinolytics, Anticoagulants are bioactive materials which act on any of the factors, cofactors, activated factors, or activated cofactors in the biochemical cascade and inhibit the synthesis of fibrin. Antiplatelet bioactive agents inhibit the adhesion, activation, and aggregation of platelets, which are key components of thrombi and play an important role in thrombosis. Fibrinolytic bioactive agents enhance the fibrinolytic cascade or otherwise aid in dissolution of a thrombus. Examples of antithrombotics include but are not limited to anticoagulants such as thrombin, Factor Xa, Factor VIIa and tissue factor inhibitors; antiplatelets such as glycoprotein IIb/IIIa, thromboxane A2, ADP-induced glycoprotein IIb/IIIa, and phosphodiesterase inhibitors; and fibrinolytics such as plasminogen activators, thrombin activatable fibrinolysis inhibitor (TAFI) inhibitors, and other enzymes which cleave fibrin.

Additionally, or alternatively, the therapeutic agents may include tyhrombolytic agents used to dissolve blood clots that may adversely affect blood flow in body vessels. A thrombolytic agent is any therapeutic agent that either digests fibrin fibres directly or activates the natural mechanisms for doing so. Examples of commercial thrombolytics, with the corresponding active agent in parenthesis, include, but are not limited to, Abbokinase (urokinase), Abbokinase Open-Cath (urokinase), Activase (alteplase, recombinant), Eminase (anitstreplase), Retavase (reteplase, recombinant), and Streptase (streptokinase). Other commonly used names are anisoylated plasminogen-streptokinase activator complex; APSAC; tissue-type plasminogen activator (recombinant); t-PA; rt-PA. While a few exemplary therapeutic agents have been listed, it will be apparent that numerous other suitable therapeutic agents may be used in conjunction with apparatus 20 and delivered through plurality of delivery ports 50.

While various embodiments of the invention have been described, it will be apparent to those of ordinary skill in the art that many more embodiments and implementations are possible within the scope of the invention. Accordingly, the invention is not to be restricted except in light of the attached claims and their equivalents. Moreover, the advantages described herein are not necessarily the only advantages of the invention and it is not necessarily expected that every embodiment of the invention will achieve all of the advantaged described.

I claim:

1. An apparatus suitable for treating a vascular condition, the apparatus comprising:
a catheter having proximal and distal regions;
a balloon disposed on the distal region of the catheter, the balloon having uninflated and inflated states, and wherein the balloon further comprises a treatment section adapted to contact the vascular condition in the inflated state; and
a plurality of delivery ports disposed along a lateral surface of the catheter at a location proximal to the balloon,
wherein the balloon is further adapted to be at least partially inflated at a location distal to the vascular condition during injection of a therapeutic agent through the plurality of delivery ports to enhance localized delivery of the therapeutic agent to the vascular condition.

2. The apparatus of claim 1 wherein the plurality of delivery ports spans a longitudinal length along the catheter that is substantially identical to a longitudinal length spanned by the treatment section of the balloon.

3. The apparatus of claim 1 wherein the balloon is adapted to perform angioplasty on the vascular condition in the inflated state.

4. The apparatus of claim 1 wherein the catheter comprises a wire guide lumen extending between the proximal and distal regions of the catheter, wherein the plurality of delivery ports are in fluid communication with the wire guide lumen, wherein the wire guide lumen is sized to receive a wire guide therein, and wherein the wire guide lumen is sized to permit injection of a therapeutic agent in an annular space between the wire guide and the catheter to thereby inject the therapeutic agent through the plurality of delivery ports.

5. The apparatus of claim 4 further comprising an inflation lumen extending between the proximal and distal regions of the catheter, wherein the inflation lumen is in fluid communication with an interior surface of the balloon.

6. The apparatus of claim 4 wherein a distal tip of the catheter comprises an inward taper configured to permit advancement of the catheter over the wire guide, while restricting flow of the therapeutic agent through the distal tip of the catheter.

7. The apparatus of claim 1 wherein at least some of the plurality of the delivery ports are angled in different directions with respect to other of the plurality of delivery ports.

8. A method suitable for treating a vascular condition, the method comprising:
providing a catheter having proximal and distal regions, a balloon disposed on the distal region, and a plurality of delivery ports disposed along a lateral surface of the catheter at a location proximal to the balloon;
inserting the catheter into a vessel with the balloon in an uninflated state;
aligning the balloon with the vascular condition and inflating the balloon to treat the vascular condition;
deflating the balloon;
advancing the catheter in a distal direction to substantially align the plurality of delivery ports with the vascular condition;
at least partially inflating the balloon at a location distal to the vascular condition; and
injecting a therapeutic agent to the vascular condition via the plurality of delivery ports.

9. The method of claim 8 wherein the plurality of delivery ports span a longitudinal length along the catheter that is substantially identical to a longitudinal length spanned by a treatment section of the balloon.

10. The method of claim 8 further comprising:
providing a wire guide lumen extending between the proximal and distal regions of the catheter, wherein the plurality of delivery of ports are in fluid communication with the wire guide lumen;
advancing the catheter over a wire guide via the wire guide lumen; and
injecting the therapeutic agent in an annular space between the wire guide and the catheter to thereby inject the therapeutic agent through the plurality of delivery ports.

11. The method of claim 8 further comprising substantially inhibiting flow of the therapeutic agent through a distal tip of the catheter.

12. The method of claim 8 further comprising using the balloon to perform angioplasty on the vascular condition in the inflated state.

13. The method of claim 8 wherein at least some of the plurality of the delivery ports are angled in different directions with respect to other of the plurality of delivery ports, the method further comprising injecting the therapeutic agent to the vascular condition at multiple different angles.

14. An apparatus suitable for treating a vascular condition, the apparatus comprising:
a catheter having proximal and distal regions;
a balloon disposed on the distal region of the catheter, the balloon having uninflated and inflated states, and wherein the balloon further comprises a treatment section adapted to contact a vessel wall in the inflated state; and
a plurality of delivery ports disposed along a lateral surface of the catheter at a location proximal to the balloon, wherein the plurality of delivery ports span a longitudinal length along the catheter,
wherein the longitudinal length spanned by the plurality of delivery ports is substantially identical to a longitudinal length spanned by the treatment section of the balloon.

15. The apparatus of claim 14 wherein the balloon is adapted to be at least partially inflated during injection of a therapeutic agent through the plurality of delivery ports to enhance localized delivery of the therapeutic agent.

16. The apparatus of claim 14 wherein the balloon is adapted to perform angioplasty on the vascular condition in the inflated state.

17. The apparatus of claim 14 wherein the catheter comprises a wire guide lumen extending between the proximal and distal regions of the catheter, wherein the plurality of delivery ports are in fluid communication with the wire guide lumen, wherein the wire guide lumen is sized to receive a wire guide therein, and wherein the wire guide lumen is sized to permit injection of a therapeutic agent in an annular space between the wire guide and the catheter to thereby inject the therapeutic agent through the plurality of delivery ports.

18. The apparatus of claim 17 further comprising an inflation lumen extending between the proximal and distal regions of the catheter, wherein the inflation lumen is in fluid communication with an interior surface of the balloon.

19. The apparatus of claim 17 wherein a distal tip of the catheter comprises an inward taper configured to permit advancement of the catheter over the wire guide, while restricting flow of the therapeutic agent through the distal tip of the catheter

20. The apparatus of claim 14 wherein at least some of the plurality of the delivery ports are angled in different directions with respect to other of the plurality of delivery ports.
